(19) United States
(12) Patent Application Publication
(10) Pub. No.: US 2012/0209359 A1
(43) Pub. Date: Aug. 16, 2012

(54) LOW-PROFILE INTRALUMINAL LIGHT DELIVERY SYSTEM AND METHODS OF USING THE SAME

(75) Inventors: James C. Chen, Bellevue, WA (US); William L. Barnard, Maple Valley, WA (US); David B. Shine, Littleton, CO (US); Jay Miazga, Seattle, WA (US)

(73) Assignee: Light Sciences Oncology Inc., Bellevue, WA (US)

(21) Appl. No.: 13/390,319

(22) PCT Filed: Aug. 13, 2010
(86) PCT No.: PCT/US10/45529

§ 371 Date: Apr. 30, 2012

Related U.S. Application Data
(60) Provisional application No. 61/234,127, filed on Aug. 14, 2009.

(51) Int. Cl. A61N 5/06

(52) U.S. Cl. 607/92

(57) ABSTRACT

A light delivery system to provide light treatment to a patient includes an elongated catheter having a light emitter that transmits light towards a target site within a patient. The catheter is sized and configured to pass through anatomical body structures to reduce or eliminate trauma associated with the delivery procedure. A visualization system of the catheter can assist a user before, during, and/or after performing light therapy. The visualization system includes sensors that provide real-time imaging or feedback.

### CROSS-REFERENCE TO RELATED APPLICATION

This application claims the benefit under 35 U.S.C. §119(e) of U.S. Provisional Patent Application No. 61/234,127 filed Aug. 14, 2009. This provisional application is incorporated herein by reference in its entirety.

### BACKGROUND

1. Technical Field

The present invention relates generally to intraluminal light delivery systems usable for medical treatment, such as light therapy.

2. Description of the Related Art

Typical light therapy employs light to treat or investigate photosensitized tissues. A photoreactive agent having a characteristic light absorption waveband is often administered to the patient, either orally or by injection or even by local delivery. The photoreactive agent is circulating through vascular system and is usually absorbed by abnormal tissue more so than by normal tissue. Once the abnormal tissue has absorbed or linked with the photoreactive agent, the abnormal tissue can be analyzed or destroyed by administering light of an appropriate wavelength or waveband corresponding to the absorption wavelength or waveband of the photoreactive agent. Even without preferential absorption, targeting of tissue can be achieved through local illumination. Vascular occlusion in the region where light is delivered leads to hypoxia and tissue kill. Light activated drug therapy has proven effective in destroying abnormal tissue, such as cancer cells, lesions, and the like. Unfortunately, traditional light therapy devices are often unsuitable for performing treatments on, for example, relatively small body structures, because these devices may cause unwanted trauma.

The objective of light therapy can be diagnostic or therapeutic, or both. In diagnostic applications, the wavelength and the intensity of light are selected to cause the photoreactive agent to fluoresce as a means to acquire information about the targeted cells without damaging the targeted cells. In therapeutic applications, the wavelength and intensity of delivered light activate the photoreactive drug causing vascular shutdown and direct cell death. In some applications, it may be difficult to deliver traditional light delivery devices into small spaces while keeping trauma at or below an acceptable level for the therapeutic application.

### BRIEF SUMMARY

Some embodiments described herein are generally related to low-profile intraluminal light delivery systems usable for performing light therapy on a subject. As used herein, the term “light therapy” is to be construed broadly to include, without limitation, methods of treating and/or diagnosing an individual using externally and/or internally applied light. In therapeutic applications, light therapy is employed to treat various types of medical conditions, such as proliferative diseases (e.g., cancer), improperly functioning anatomical features, and conditions associated with, for example, infections, allergies, autoimmunity problems, and the like. Anatomical features can include, but are not limited to, systems of the body (e.g., the vascular system, lymphatic system, or skeletal system), structures of the body (e.g., organs such as the liver), tissue, cells, and the like. Muscle, bone, cartilage, connective tissue, organs (e.g., lymphoid organs), body vessels (e.g., blood vessels, bile ducts, and the like), and glands are exemplary body structures that can be conveniently treated using one or more light delivery systems.

Based on the condition to be treated, a target site can be destroyed, reduced, stimulated, or otherwise treated to elicit a desired response. To destroy unwanted tissue, the light delivery system can emit energy that causes cell damage or destruction via, for example, necrosis and/or apoptosis. The light delivery system, in some embodiments, can treat a target site of tissue in order to promote tissue growth (e.g., cell division, cell growth or enlargement, and the like), increase the rate of healing, improve bodily functioning, reduce or minimize pain, relieve stiffness, and the like. The treatments can control or effect scarring, tissue augmentation, tissue regeneration, tissue reduction, and the like. For example, the target site can be treated to reduce, limit, or otherwise prevent scarring. The subject's immune system can also be affected (e.g., up regulated, down regulated, cycled between up regulation and down regulation, and the like) using the light delivery system. Inflammation and other unwanted conditions can also be controlled using the light delivery system. Accordingly, a wide range of procedures can be performed with the light delivery system to treat various types of conditions.

To access a target site, the light delivery system can be configured and dimensioned to pass through various naturally occurring anatomical features, such as conduits (e.g., blood vessels). The light delivery system can be delivered to an internal site in the subject without causing an appreciable amount of trauma, thereby reducing, limiting, or substantially preventing injury, bleeding, and infection, as well as other types of trauma or secondary conditions attributable to the light therapy procedure. For example, an elongated catheter of the light delivery system can be delivered to a remote internal target site percutaneously or using naturally occurring orifices and physiological conduits.

The vascular system provides routes (venous routes and arterial routes) suitable for accessing numerous sites within an individual's body. Various access or entry sites can provide access to the vascular system. The antecubital fossa, inguinal region, carotid, and other access sites can provide convenient access to the vascular system, which may deliver blood to targeted tissue, such as tumors. The vessels of the vascular system that supply blood to the tumor can be used to access and to treat the tumor.

An intravascular light delivery system can include a low-profile catheter sufficiently flexible for delivery along a tortuous delivery path through the vascular system. The access site for the catheter can be the same access sites used for other procedures, such as angioplasty. A single access site can thus provide access for performing light therapy and a separate procedure, which may or may not be related to the light therapy.

The elongated catheter includes one or more light sources capable of emitting energy for light therapy. The number, types, and positions of the light sources can be selected based on the light therapy to be performed. The light sources may include, without limitation, one or more laser sources, light emitting diodes (LEDs), electroluminescent material, and other types of incandescent, halogen, fluorescent, phosphor, and electroluminescent sources. LEDs can be, without limitation, polymeric light emitting diodes, organic light emitting diodes, metallic light emitting diodes, and/or combinations thereof. Additionally or alternatively, the elongated catheter can include one or more optical guides (e.g., a single fiber optic or a bundle of fiber optics). Optical guides can transmit light from an external light source through the elongated catheter to the target site.

The light delivery system can have an elongated catheter that includes one or more steering elements. As used herein, the term “steering element” is broadly construed to include, without limitation, one or more fins, tabs, flow inhibitors, or other structures that cooperate or interact with fluid flow to move or position at least a portion of an elongated catheter. Steering elements can provide lift, drag, or other forces for controlling or affecting directional stability. Additionally or alternatively, a steering element can have a generally straight configuration, arcuate configuration, or any other configuration suitable for providing the desired steerability.

One or more sensors for detecting or imaging can be coupled (either directly or indirectly) to the elongated catheter. For example, a sensor can be bonded, affixed, embedded, incorporated, or otherwise coupled to the elongated catheter.

In some embodiments, an intraluminal system for performing light therapy is provided. The intraluminal system may include an elongated catheter having a proximal section, a distal section, and a central section between the proximal section and the distal section. The central section and the distal section are configured and dimensioned to be delivered through a lumen of a vessel, such as a peripheral vessel. The vessel may be in the form of a vein, an artery, a venule, an arteriole, a capillary, a lymphatic vessel or channel, and the like. The distal section includes at least one light emitter operable to output a sufficient amount of light to perform light therapy on tissue. The tissue, in some embodiments, is adjacent or proximate to the vessel.

In some embodiments, a method of treating target tissue of a subject is provided. The method includes moving a catheter along a lumen of a vascular vessel. A light emitter of the catheter is positioned in the vascular vessel based on a position of the target tissue. The light emitter is activated to deliver a therapeutically effective amount of light to the target tissue, which in some embodiments is on the outside of the vascular vessel. The target tissue may be cancerous tissue or a lesion (e.g., a localized abnormal tissue in a body part) proximate to or abutting the catheter. In some embodiments, activating the light emitter comprises sequentially increasing light emitted from the catheter. In some embodiments, activating the light emitter comprises outputting a substantially constant amount of light energy from the catheter.

In other embodiments, a method for performing light therapy on a subject includes moving a catheter along a first lumen of a vascular system towards a bifurcated section of the vascular system. The bifurcated section includes a second lumen and a third lumen. The second and third lumens are angled with respect to the first lumen. The catheter is positioned along the first lumen such that a distal tip of a catheter moves laterally in response to at least one steering element of the catheter interacting with blood flow through the first lumen. The laterally displaced distal tip is then moved through the bifurcated section and into the second lumen. A light emitter of the catheter is activated to illuminate tissue adjacent the second lumen.

In some embodiments, a light delivery system includes a first catheter, a second catheter, and a control system. The first catheter includes a first light emitter and a first sensor. The second catheter includes a second light emitter and a second sensor. The first sensor is capable of detecting light emitted by the second light emitter. The second sensor is capable of detecting light emitted by the first light emitter. The control system is configured to control the first light emitter based on a signal from the second sensor and to control the second light emitter based on a signal from the first sensor.

A method for performing light therapy, in some embodiments, includes positioning a first catheter in a subject. The first catheter includes a first light emitter and a first sensor. A second catheter is positioned in the subject. The second catheter includes a second light emitter and a second sensor. The second light emitter is capable of emitting light detectable by the first sensor. The second sensor is capable of detecting light emitted by the first light emitter. The second catheter is positioned with respect to the first catheter such that the first sensor detects light emitted by the second light emitter when the second light emitter is activated. The second sensor detects light emitted by the first light emitter when the first light emitter is activated.

In some embodiments, an intraluminal catheter for performing light therapy on a lymph node includes a central section and a distal section. The central section is configured for placement in a subject. The distal section is coupled to the central section. The distal section includes at least one light source capable of outputting light for performing light therapy. The distal section is configured and dimensioned for delivery through a lumen of a lymph or blood vessel to position the at least one light source within range of lymphatic tissue.

Some embodiments of treating lymphatic tissue include moving a catheter along a body lumen towards lymphatic tissue. A distal tip of the catheter is advanced through the body lumen towards the lymphatic tissue. A light emitter of the catheter is activated to deliver light to the lymphatic tissue adjacent the light emitter.

In some embodiments, a catheter for performing light therapy is provided. The catheter includes a catheter body for placement in a subject, a light emitting system coupled to the catheter body, and a detector system coupled to the catheter body. The detector system is configured to detect a characteristic of tissue illuminated by light from the light emitting system, and also to send at least one signal based on the detected characteristic of the tissue. In some embodiments, the catheter includes a control system configured to receive the at least one signal from the detector system and to provide an output based on the received signal.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles may not be drawn to scale, and some of these elements may be arbitrarily enlarged and positioned to improve drawing legibility.

FIG. 1 shows a light delivery system having a distal section positioned in a body structure, according to one illustrated embodiment.

FIG. 2 is a side elevational view of a distal section of a light delivery system positioned in a body vessel and performing light therapy, according to one illustrated embodiment.

FIG. 3 is a front view of the distal section of the light delivery system of FIG. 2.

FIG. 4 is a side elevational view of a light delivery system, according to one illustrated embodiment.

FIG. 5 is a cross-sectional view of the distal section of the light delivery system of FIG. 4, according to one illustrated embodiment.

FIG. 6 shows a distal section of a light delivery system spaced from a blood vessel extending through a tumor, according to one illustrated embodiment.

FIG. 7 shows the distal section of the light delivery system moving through the blood vessel towards the tumor.

FIG. 8 shows the distal section of the light delivery system of FIG. 7 positioned within the tumor.

FIG. 9 is a side elevational view of a distal section of a light delivery system including a plurality of steering elements, according to one illustrated embodiment.

FIG. 10 is a plan view of the distal section of the light delivery system of FIG. 9.

FIG. 11 is a front elevational view of the distal section of FIG. 9.

FIG. 12 shows the distal section of the light delivery system of FIG. 9 positioned in a vascular body vessel upstream of a bifurcated section of the vascular system, in accordance with one illustrated embodiment.

FIG. 13 shows a distal tip of the distal section of FIG. 12 positioned adjacent the upstream body vessel.

FIG. 14 shows the distal section of FIG. 12 advancing through a downstream body vessel of the bifurcated section.

FIG. 15 shows a steerable light delivery system in a vascular vessel, according to one illustrated embodiment.

FIG. 16 illustrates a steerable distal section of a light delivery system, according to one illustrated embodiment.

FIG. 17 shows the distal section of FIG. 16 when fluid flows thereby, according to one illustrated embodiment.

FIG. 18 is a side elevational view of a selectively actuatable distal section, according to one illustrated embodiment.

FIG. 19 is a longitudinal cross-sectional view of the distal section of FIG. 18 taken along the line 19-19, according to one illustrated embodiment.

FIG. 20 shows a longitudinal cross-section of a distal section of a magnetically steerable elongated catheter and an external steering device, according to one illustrated embodiment.

FIG. 21 is a side elevational view of a distal section of a magnetically steerable elongated catheter, according to one illustrated embodiment.

FIG. 22 shows a distal section of an elongated catheter in a vessel, the distal section includes a propulsion system, according to one illustrated embodiment.

FIG. 23 is a side elevational view of a distal section of an elongated catheter with a propulsion system, according to one illustrated embodiment.

FIG. 24 is a side elevational view of a distal section of an elongated catheter with a propulsion system, according to one illustrated embodiment.

FIG. 25 shows a distal section of a light delivery system within a lymphatic vessel connected to a lymph node, according to one illustrated embodiment.

FIG. 26 shows the light delivery system of FIG. 25 extending through the lymphatic vessel into the cortex of the lymph node.

FIG. 27 shows the light delivery system of FIG. 25 emitting light through tissue of the lymph nodes, according to one illustrated embodiment.

FIG. 28 shows an insertion device positioning a light delivery system in tissue, according to one illustrated embodiment.

FIG. 29 is a side elevational view of internal components of a distal section of an elongated catheter, according to one illustrated embodiment.

FIG. 30 is a side elevational view of a distal section of a light delivery system having a light emitter and an external detector, according to one illustrated embodiment.

FIG. 31 is a side elevational view of a distal section of a light delivery system, wherein the distal section includes a working lumen and a light emitter interposed between a pair of detector systems, according to one illustrated embodiment.

FIG. 32 shows a detector system, according to one illustrated embodiment.

FIG. 33 is an elevational view of a distal section of a light delivery system.

FIG. 34 is an elevational view of the distal section of FIG. 33 in a curved configuration.

### DETAILED DESCRIPTION

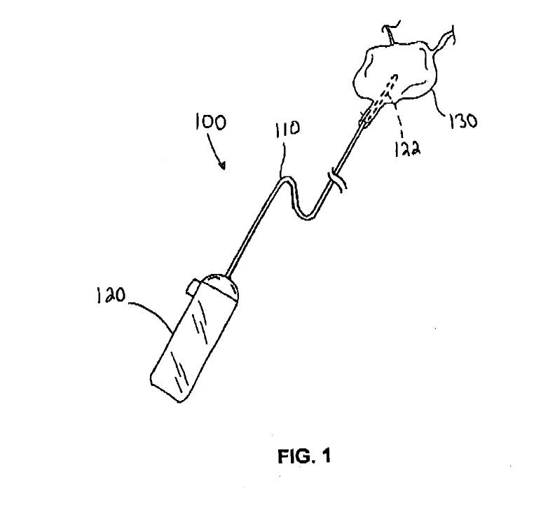

FIG. 1 shows a light delivery system 100 including a control system 120 and an intraluminal elongated catheter 110 coupled to the control system 120. A portion 122 of the elongated catheter 110 is positioned in a patient's body structure 130. Once positioned, the elongated catheter 110 can emit energy to treat targeted tissue. It is understood that even if one cell is “targeted,” it is possible that other cells in the vicinity of the targeted cell may also be treated (e.g., subjected to light). The user can use the control system 120 to control various operating parameters, such as energy variation, energy intensity, activation program, or the position of the elongated catheter 110, as well as other operating parameters that may affect the patient.

The light delivery system 100 can be delivered through various types of anatomical features in order to access the target site. The elongated catheter 110 may be delivered through arterial vessels or venous vessels, or both, and into the body structure 130. The elongated catheter 110 can be navigated through interior regions of the body structure 130, which may require navigating through a series of branch sections of the vascular system.

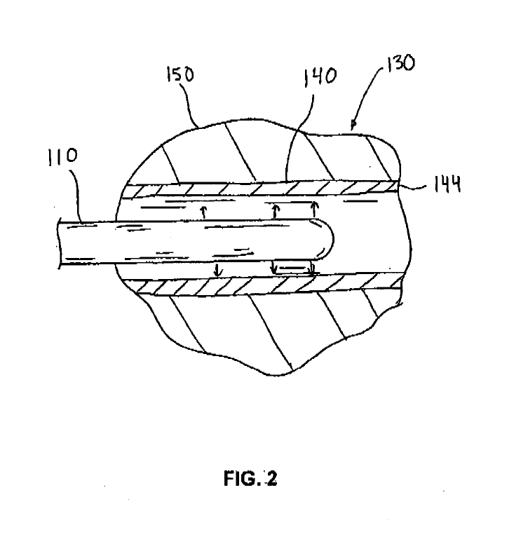

FIGS. 2 and 3 show the elongated catheter 110 positioned within a body vessel or duct 140 extending through the body structure 130. Emitted light (represented by arrows) is transmitted through a wall 144 of the vessel 140. The light is then transmitted through the tissue 150 surrounding the vessel 140 in order to illuminate the targeted tissue.

A treatment agent for light therapy can include, but is not limited to, one or more photoreactive agents, photosensitizing agents, or other types of treatment agents having at least one characteristic light absorption waveband and that can be administered to the patient, either orally or by injection or even by local delivery to the treatment site. The treatment agent can be selectively absorbed by targeted tissue (e.g., abnormal tissue). Once the targeted tissue has absorbed the treatment agent, the targeted tissue is treated (e.g., destroyed) by administering light of an appropriate wavelength or waveband corresponding to the absorbance wavelength or waveband of the treatment agent. For example, abnormal regions of the tissue 150 of FIGS. 2 and 3 can be selectively treated (e.g., destroyed) without treating healthy tissue 150. The healthy tissue 150 can remain substantially intact in order to continue functioning. The wall 144 may also have unwanted tissue, such as cancer cells. At least a portion of the tissue 150 can consist of healthy cells. Abnormal cells of the wall 144 can be selectively targeted with the treatment agent. The selectively targeted tissue 144 is then illuminated and destroyed using the elongated catheter 110.

Alternatively, a region in the vicinity of the elongated catheter 110 can be treated after drug administration without waiting for substantial drug accumulation in the abnormal tissue. In that case, the tissue destruction may result from vessel occlusion in the region defined by light penetration.

FIG. 3 shows a target site 152 adjacent the vessel 140. The elongated catheter 110 can deliver a sufficient amount of light to the target site 152 to elicit a desired response. The power density provided by the elongated catheter 110 can be greater than about 0.5 mW/cm². In some embodiments, the energy density is equal to or greater than about 2 mW/cm², 10 mW/cm², 30 mW/cm², 40 mW/cm², or 50 mW/cm², or ranges encompassing such energy densities. In some embodiments, for example, the elongated catheter 110 output having an average power density of at least 2 mW/cm² at a wavelength between approximately 630-730 nm. The light intensity can be generally constant or varied (e.g., gradually increased or decreased, or both) to enable apoptosis, necrosis, and the like. To treat a relatively large treatment site (e.g., the prostrate or other organ), the light intensity is sequentially increased to selectively induce apoptosis and/or necrosis of cancerous tissue.

Even though the target site 152 is spaced a distance D from the elongated catheter 110, the elongated catheter 110 can deliver a therapeutically effective amount of light to the target site 152. The distance D can be at least about 0.5 mm, 5 mm, 10 mm, 20 mm, 50 mm, 10 cm, or 20 cm, or ranges encompassing such distances. A user can determine the amount of energy sufficient to effectively treat the target site 152. In some embodiments, the user can control or otherwise program the elongated catheter 110 based on the determined amount of energy. In some embodiments, a substantial portion of the target tissue 152 is treated with the treatment agent and subsequently illuminated without adversely affecting a selected portion (e.g., a significant portion) of healthy tissue in the vicinity of the targeted tissue. For example, the elongated catheter 110 can treat the remote target site 152 outside of the vessel 140 without damaging the vessel wall 144.

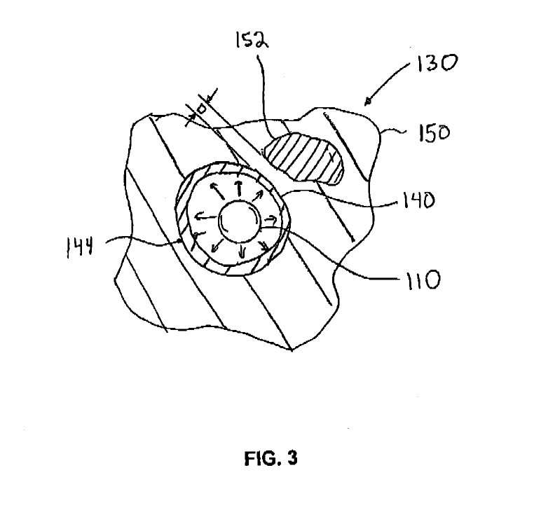

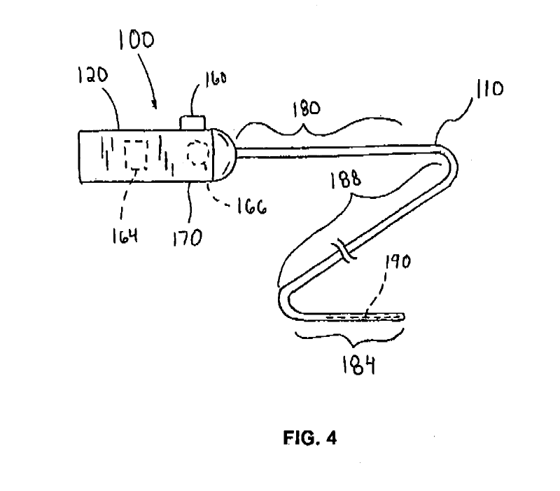

Once a desired response is achieved, the elongated catheter 110 can be conveniently removed from the subject by retracting the catheter 110 through the same body features used to access the body structure 130. Because the elongated catheter 110 is flexible, it can be withdrawn through the vascular system without causing damage (e.g., significant damage) to the vascular vessels, as well as other body features near the removal path. Referring to FIG. 4, the control system 120 can be indirectly or directly coupled to the elongated catheter 110. The control system 120 includes a controller 160 for user input and/or user output and a power supply 164 (shown in phantom) in communication with the elongated catheter 110. The controller 160 can be operated to select the amount of energy emitted by the elongated catheter 110. A housing 170 surrounds and protects the power supply 164 and, in some embodiments, can be comfortably gripped by a user.

The illustrated power supply 164 of FIG. 4 is a battery, such as a lithium battery. In other embodiments, the light delivery system 100 is powered by a supercapacitor. In other embodiments, the light delivery system 100 is powered by an AC power source, such as an electrical outlet typically found at a hospital, medical facility, residence, or other suitable location for performing light therapy. The control system 120 can include a power cord that can be connected to the AC power source. Accordingly, various types of internal and/or external power sources can be utilized to power the light delivery system 100. External power sources can be directly or indirectly coupled to the control system 120 or other component(s) of the delivery system 100. In some embodiments, an external power source is inductively coupled to the light delivery system 100 to power electrical components. For example, the external power source can charge an internal power source, such as a rechargeable battery.

In some embodiments, the controller 160 includes one or more displays, keyboards, touch pads, control modules, or any peripheral user input devices and/or output devices. For example, the controller 160 can include a rotatable dial for selecting an output of a light emitter 190 and a separate screen for displaying progress of the therapy.

The control system 120 can include an actuation mechanism 166 (shown in phantom) used to rotate, deflect, actuate, extend, and/or retract the elongated catheter 110. The actuation mechanism 166 can be manually or automatically operated. In some embodiments, the actuation mechanism 166 includes one or more motors, reels, pulley systems, braking systems, and/or cables used to operate the elongated catheter 110. For example, a motor can drive a pulley system that has a plurality of cables running through the catheter. The catheter is moved when at least one of the cables is pulled proximally.

The control system 120 may include, without limitation, one or more sub controllers, processors, microprocessors, digital signal processors (DSP), application-specific integrated circuits (ASIC), and the like. To store information, the control system 120 may also include one or more storage devices. The storage devices can be in the form of volatile memory, non volatile memory, read only memory (ROM), random access memory (RAM), and the like.

With continued reference to FIG. 4, the elongated catheter 110 includes a proximal section 180, a distal section 184, and a central section 188 extending between the proximal section 180 and the distal section 184. The distal section 184 has the light emitter 190 (illustrated in broken lines) operable to output light suitable for performing light therapy. The distal section 184 and central section 188 are configured and dimensioned to be delivered through anatomical features to position the emitter 190 within range of the target site. The illustrated proximal section 180 is coupled directly to and extends distally from the control system 120.

The length of the catheter 110 can be selected based on the length of a delivery path between the access site and the target site. If the elongated catheter 110 is delivered along a long windy delivery path, the central section 188 can be flexible so as to assume various configurations that facilitate delivery. The flexibility of the catheter 110 can be selected to reduce or limit the likelihood of permanent kinking, which may cause the elongated catheter 110 to become trapped in the subject.

To deliver power from the power supply 164 to the light emitter 190, one or more electrical components (e.g., wires, electrical connectors, conductive strips, and the like) can extend proximally from the light emitter 190 to the power supply 164. If the light emitter 190 is an addressable array, the operator can select the length of the addressable array for outputting light. The selected length of the addressable array can be approximately equal to the length of the target site. For example, the entire addressable array can be activated to treat a large treatment site, such as a large tumor. If the treatment site is small, the user can turn ON only a section of the addressable array. The control system 120 can store different programs for activating different sections of the addressable array based on, for example, input from the user. The addressable array can be a light bar (e.g., a single-sided light bar, a double-sided light bar, or the like) with spaced apart independently activatable light sources or other type of addressable light emitting device.

The cross-sectional width of the elongated catheter 110 can be increased or decreased by increasing or decreasing the size of these electrical components. Various types of power switches and transistors can be used to control electrical components of the elongated catheter 110, and may further reduce the size of the distal section 184. Miniature power switches, for example, can simultaneously or individually operate one or more components of the light emitter 190.

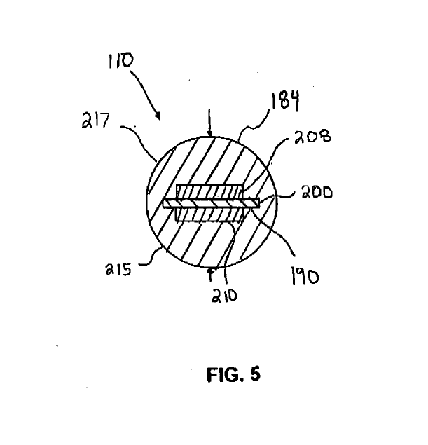

Referring to FIGS. 4 and 5, the light emitter 190 can output a therapeutically effective amount of light to treat a selected amount of tissue in a selected amount of time. The light emitter 190 can output a sufficient amount of light to perform light therapy on tissue adjacent (e.g., near or proximate) the vascular vessel. A ratio of power density (in units of mW/cm²) to the axial cross-sectional area of the distal section 184 (in units of cm²) can be equal to or greater than about 50 mW/(cm²)². The power density may be less than about 50 mW/cm². In some embodiments, the power density is in the range of about 1 mW/cm² to about 50 mW/cm². Other power densities are also possible. The total energy dose can be equal to or less than 400 J, 600 J, or 800 J per treatment. Such embodiments are especially well suited for passing through vessels with small diameters (e.g., diameters equal to or less than about 0.1 cm). The total energy dose may be in the range of about 1 J/cm² to about 800 J/cm² per treatment depending on the amount and type of tissue to be treated. Other total energy doses are also possible. In some embodiments, a ratio of power density to axial cross-sectional area of the distal section 184 is equal to or greater than about 500 mW/(cm²)². Such embodiments are especially well suited for passing through vessels with extremely small diameters (e.g., peripheral blood vessels with diameters equal to or less than about 200 μm) and treating target tissue a significant distance from the vessels. In some embodiments, a ratio of power density to axial cross-sectional area of the distal section 184 is equal to or greater than about 1,000 mW/(cm²)² to treat tissue spaced from the distal section 184. The catheter 110 can have energy sources coupled to one side of a substrate to provide these types of ratio or power density. Such single-sided light bars can have relatively small dimensions while being capable of outputting high amounts of energy. In some embodiments, the ratio of power density to axial cross-sectional area of the distal section 184 is within a range of about 500 mW/(cm²)² to about 2,000 mW/(cm²)². U.S. application Ser. No. 12/445,061 discloses various types of single-sided light bars, light source mounting techniques, and components that can be utilized to manufacture the distal section 184. By way of example, the distal section 184 can have a clear substrate carrying an array of light sources on one side of the substrate such that, when the light sources are energized, light is emitted outwardly from both sides of the substrate. U.S. application Ser. No. 12/445,061 is incorporated by reference in its entirety.

In some embodiments, a ratio of power density to axial cross-sectional area of the distal section 184 is in the range of about 10 mW/(cm²)² to about 200 W/(cm²)². In some embodiments, at least a portion of the distal section 184 has a ratio of power density to axial cross-sectional area greater than about 200 mW/(cm²)² for passing along lumens of medium sized vascular vessels. Other energy densities are also possible, if needed or desired. Various types of light bars, light sources, such as laser diodes, ultrabright LEDs, phosphor elements (e.g., fibers coated with phosphor), and the like can be used to achieve small dimensions with a relatively high energy output.

The longitudinal length of the light emitter 190 can be increased or decreased to increase or decrease the length of tissue illuminated by the emitter 190. The light emitter 190 of FIG. 5 is a double-sided light bar, including a substrate 200, upper light sources 208, and lower light sources 210. The upper light sources 208 are coupled to an upper surface of the substrate 200, and the lower light sources 210 are coupled to a lower surface of the substrate 200. An encapsulant 215 surrounds and protects the embedded light emitter 190. Either the upper light sources 208 or the lower light sources 210 can be eliminated to form a single-sided light bar, such as those disclosed in U.S. application Ser. No. 12/445,061.

Various types of mounting techniques can be utilized to couple the light sources 208, 210 to the substrate 200. U.S. Pat. Nos. 5,445,608; 5,800,478; 5,876,427; application Ser. No. 10/888,567 (corresponding to U.S. Publication No.: US 2005/0128742 A1) and U.S. Application Serial No. 12/445,061 disclose catheter designs, distal tips, expandable members, light emitters (including light sources, light bars, and light sources), mounting arrangements of light sources, electrically conducting substrates, and electrical components, as well as other features (e.g., expandable members such as balloons), materials, and devices that can be applied to or used in connection with one or more of the embodiments, features, systems, devices, materials, methods, and techniques discussed herein. U.S. Pat. Nos. 5,445,608; 5,800,478; 5,876,427; and application Ser. No. 10/888,567 are incorporated by reference in their entireties.

To facilitate delivery through small spaces, the elongated catheter 110 of FIG. 5 can have a lubricious coating 217. The lubricious coating 217 reduces frictional forces when the catheter 110 engages tissue. The coating 217 can comprise one or more polymers, such as nylon, TEFLON®, polytetrafluoroethylene (PTFE), or other biocompatible materials. The coating 217 can be disposed on an outer surface of the encapsulant 215. Depositing, impregnating, spraying, flow coating, or other fabrication techniques are suitable for forming the coating 217. Different types of biocompatible materials can be used to make the coating 217, as well as other components of the catheter 110.

FIG. 6 shows the elongated catheter 110 ready for delivery through a body vessel 230 and into a target site 235, illustrated as a tumor or lesion. In general, an atraumatic distal tip 220 can be navigated and advanced distally through a lumen 240 of the vessel 230 (see FIGS. 6 and 7). After the distal section 184 is within range of the targeted tissue, the light emitter in the distal section 184 can be activated. As shown in FIG. 8, light (represented by arrows) can be transmitted through a wall 260 of the body vessel 230 and through the surrounding tissue 270. The distal section 184 can be positioned at any point along the body vessel 230 to treat the tissue 270 in stages or en bloc.

Different types of procedures may provide access to the tumor 235. For example, minimally invasive procedures, open procedures, semi-open procedures, or other surgical procedures can provide access to body structures that can define an appropriate delivery path to the tumor 235. The treatment agent (e.g., talaporfin sodium) can be administered to the tumor 235 by a suitable delivery means. To deliver a therapeutically effective amount of the treatment agent, the treatment agent can be administered intravenously or by other suitable means, including, without limitation, local delivery or systematic delivery, or both. After the treatment agent is adequately dispersed at the target site, the light delivery system 100 is used to activate the treatment agent. For example, the light emitter 190 of FIG. 8 can illuminate the sensitized target site for a sufficient amount of time to perform the desired light therapy. The treated tissue may break down (e.g., immediately or gradually over an extended period of time) and may subsequently be absorbed by the subject's body. In this manner, unwanted tissue can be destroyed, reduced in size, or otherwise treated to improve the health of the patient. U.S. Pat. Nos. 5,308,861; 6,554,853; 6,602,274; 7,018,395; 7,053,210; 6,344,050; 6,899,723; 5,997,842; and Reissue Pat. No. 37,180, which are incorporated by reference in their entireties, disclose various treatment agents and methods of using the same.

With continued reference to FIGS. 6-8, the elongated catheter 110 can be actively and/or passively advanced through the body vessel 230. The elongated catheter 110, in some embodiments, can be passively delivered through the body vessel 230 using natural functioning of the subject's body. For example, the illustrated distal section 184 is advanced distally using natural blood flow 264 (see FIG. 7). The blood flow 264 can pull the elongated catheter 110 downstream through the vessel 230. A guide wire, delivery sheath, introducer, delivery needle, or other delivery device can be used to deliver the catheter 110.

Various types of visualization techniques (including either internal visualization techniques or external visualization techniques, or both) can be used to help deliver, position, operate, and/or remove the light delivery system 100. For example, ultrasound, fluoroscopy, CT, MRI, combinations thereof, or other imaging techniques and associated equipment can help evaluate an access site, delivery path, target tissue, effect of light therapy, and position of the elongated catheter 110 before, during, and/or after light therapy. Visualization may assist a user navigating the elongated catheter 110 along a delivery path to reduce, limit, or substantially prevent trauma to the subject. U.S. Pat. Nos. 6,138,681; 6,210,425; 6,238,426; which are incorporated by reference in their entireties, disclose various types of visualization techniques, systems, devices, and/or components capable of viewing at least a portion of the light delivery system 100. Various types of other tracking systems, such as magnetic or RF systems, can be employed. These types of systems can have one or more electromagnetic transponders, magnetic field generators, RF coils, and the like used to track any component(s) of interest. Different types of transponders can be incorporated into the disclosed embodiments for different types of external tracking systems. One conventional tracking system suitable for use with at least some of the disclosed embodiments is from CALYPSO® Medical in Seattle, Wash.

As noted above, the illustrated tumor 235 of FIGS. 6-8 can be accessed via the naturally occurring body vessel 230 to reduce, limit, or substantially prevent ancillary damage to the patient, even when the vessel 230 is somewhat narrow. For example, the elongated catheter 110, or at least a portion thereof, can have a cross-sectional width that is less than about 1.25 mm. In such embodiments, the distal section 184 can be conveniently passed through somewhat small blood vessels. The elongated catheter 110 can have a cross-sectional width that is less than about 1 mm. In such embodiments, the distal section 184 can be conveniently delivered through branching sections of the vascular system, even rapidly branching sections, to reach target sites at or near remote or peripheral vessels. In some embodiments, the elongated catheter 110 has a cross-sectional width that is less than about 0.1 mm to access and to pass through vessels or nodes of the lymphatic system. To perform light therapy on the respiratory system, the elongated catheter 110 is sized for delivery along intrapulmonary airways. To treat an inferior lobe of a lung, for example, the elongated catheter 110 is passed down the trachea and through the right main bronchus into the inferior lobar bronchus. The elongated catheter 110 is moved through branching sections of the bronchioles to the target site. In order to perform light therapy at different locations, the elongated catheter 110 can be retracted and advanced any number of times to reposition the distal section 184.

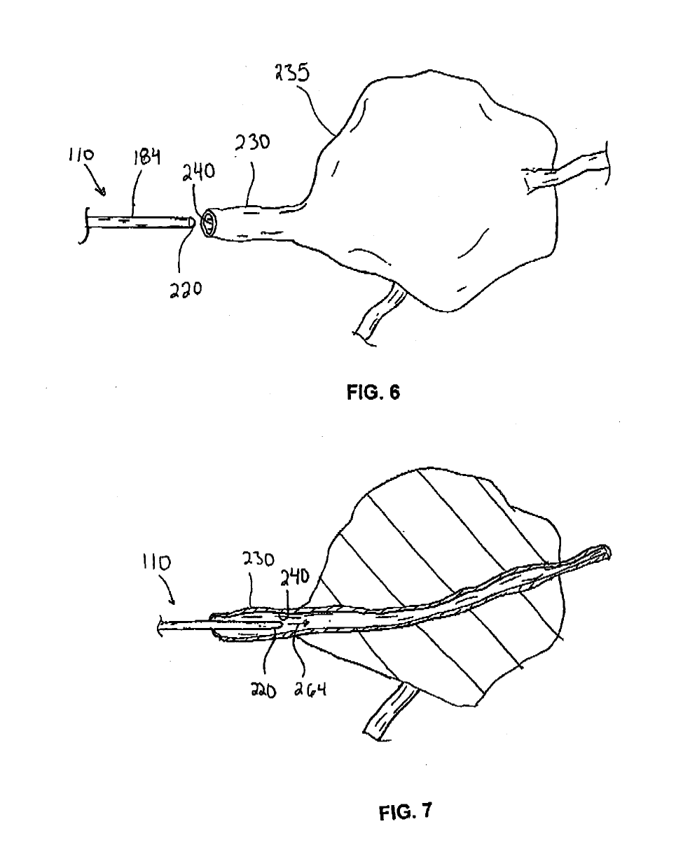

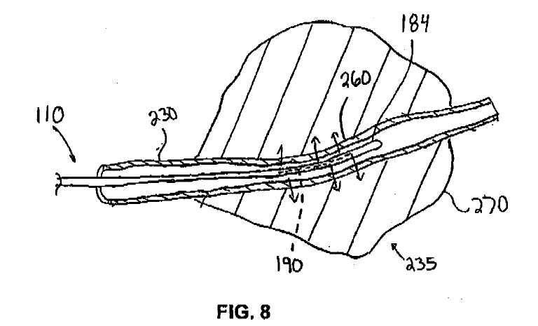

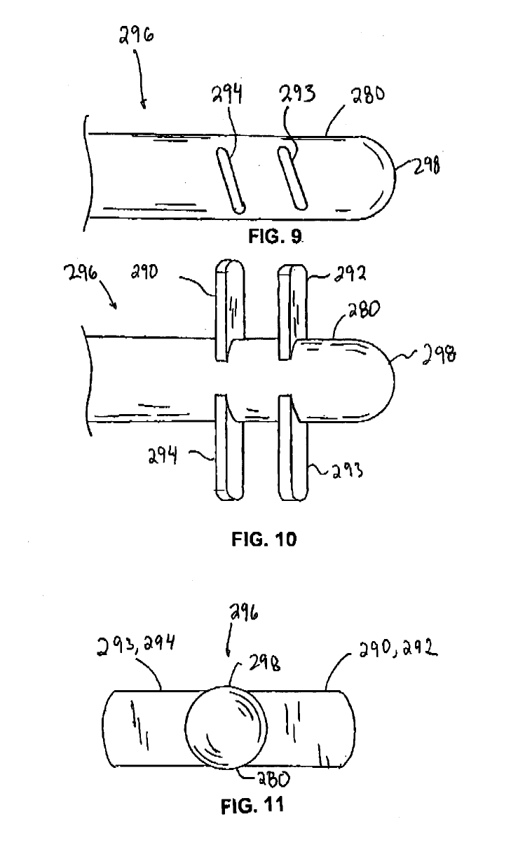

FIGS. 9-11 show a distal section 280 of an elongated catheter 296 with steering elements 290, 292, 293, 294. The steering elements 290, 292, 293, 294 are used to advance, position, and/or steer the distal section 280 by interacting with fluid flow (e.g., blood flow, lympathic fluid, airflow, and the like).

With continued reference to FIG. 10, a main body 298 of the elongated catheter 296 is interposed between the pair of steering elements 290, 292 and the pair of steering elements 293, 294. The steering elements 290, 292 are diametrically opposed to the steering elements 294, 293, respectively. The number, configurations, and positions of the steering elements can be selected based on the mechanical properties (e.g., the flexibility) of the main body 298, length and configuration of the delivery path, and the size of the body vessel through which the elongated catheter 296 is to be delivered. U.S. Pat. Nos. 6,658,278 and 6,925,320, which are both incorporated by reference in their entireties, disclose various types of steering elements (e.g., fins) and methods of navigating a catheter. These can be used in connection with the light delivery systems disclosed herein.

The steering elements 290, 292, 293, 294 can be deployable to allow fluid flow past the catheter 296 without appreciably deflecting the main body 298. Actuators (e.g., mechanical actuators or pneumatic actuators) can be used to deploy and retract the steering elements 290, 292, 293, 294. Piezoelectric materials, shape memory materials, or combinations thereof may also be used to retract/extend the steering elements 290, 292, 293, 294. In other embodiments, the steering elements 290, 292, 293, 294 can lay along the outside of the main body 298 to keep the main body 298 in an undeflected configuration. The steering elements 290, 292, 293, 294 can extend outwardly to move the main body 298 to a deflected configuration.

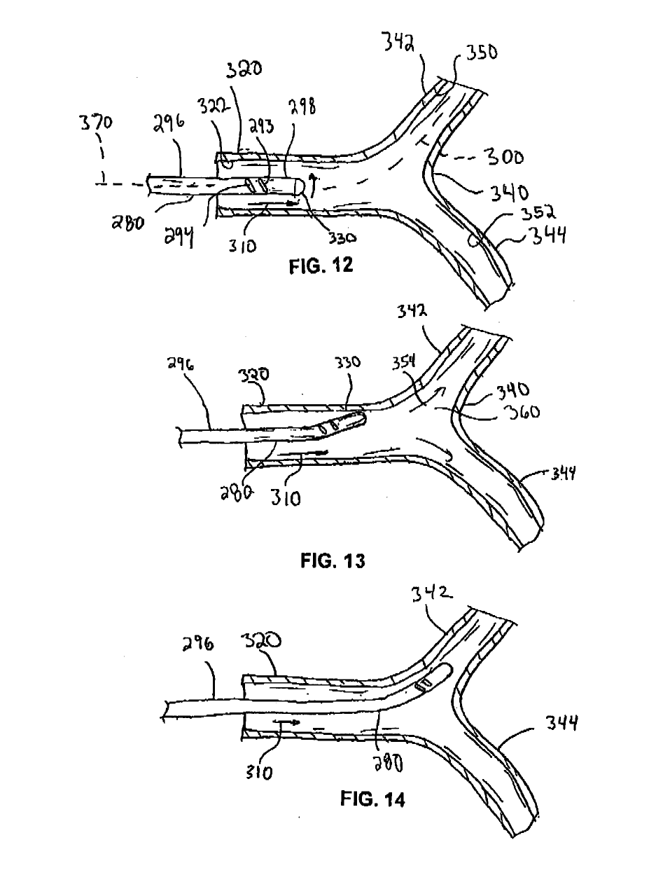

FIGS. 12-14 illustrate the elongated catheter 296 delivered along a non-linear delivery path 300 (shown in phantom in FIG. 12). The steering elements 290, 292, 293, 294 are configured to move the main body 298 into a selected configuration to facilitate delivery along the delivery path 300. The illustrated steering elements 290, 292, 293, 294, for example, urge the main body 298 into a substantially arcuate configuration (see FIG. 13) in response to blood flowing distally (indicated by the arrow 310) through a first vessel 320. Blood 310 acts on the angled surfaces of the steering elements 290, 292, 293, 294 causing the steering elements and associated main body 298 to move into the desired configuration. For example, the blood flow 310 can interact with the steering elements 290, 292, 293, 294 to drive a distal tip 330 of the elongated catheter 296 outwardly towards a wall of the first vessel 320.

In one method of traveling through a branching section, the elongated catheter 296 is moved through the upstream first vessel 320 towards a downstream Y-shaped bifurcated section 340. The bifurcated section 340 includes a second vessel 342 and a third vessel 344 angled with respect to the second vessel 342.

The elongated catheter 296 can be rotated about its longitudinal axis 370 (FIG. 12) to rotationally position the steering elements 290, 292, 293, 294 so as to laterally position the distal section 280 in response to the steering elements 290, 292, 293, 294 redirecting the flow of blood. The laterally displaced distal tip 330 shown in FIG. 13 is moved downstream towards a junction 360 of the bifurcated section 340. The distal tip 330 is then advanced through the junction 360 and into the second vessel 342, as indicated by the arrow 354. The elongated catheter 296 of FIG. 14 is then advanced downstream through the second vessel 342. In this manner, the elongated catheter 296 can be easily advanced through any number of branching sections of the vascular system, or any other branching body structures of the subject.

To advance the elongated catheter 296 through the third vessel 344, the elongated catheter 296 of FIG. 12 can be rotated about its longitudinal axis 370 about 180°. The steering elements 290, 292, 293, 294 interact with the blood flow so as to push the distal tip 330 towards the lower side of the vessel 320. The elongated catheter 296 may then be advanced distally towards and through the junction 360 and into the third vessel 344.

The orientation, longitudinal positions, and number of the steering elements can be selected based on the configuration of the bifurcated section 340. In some embodiments, the elongated catheter 296 is configured to navigate through the bifurcated section 340 in which a second lumen 350 and a third lumen 352 form an angle equal to or less than about 10°, 45°, 60°, 90°, 130°, or 170°, or ranges encompassing such angles. In some embodiments, the elongated catheter 296 in the first lumen 322 is configured to navigate into the second lumen 350, wherein the lumens 322, 350 define an angle equal to or less than about 45°, 60°, 90°, 130°, 170°, or ranges encompassing such angles. In some embodiments, the first lumen 322 and the second lumen 350 define an angle less than about 140°.

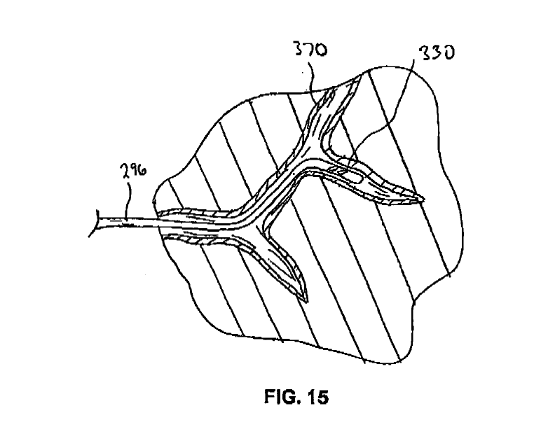

FIG. 15 illustrates the elongated catheter 296 positioned within a branching section of a vascular system 370. The elongated catheter 296 can be delivered through any number of branching sections to reach the target site.

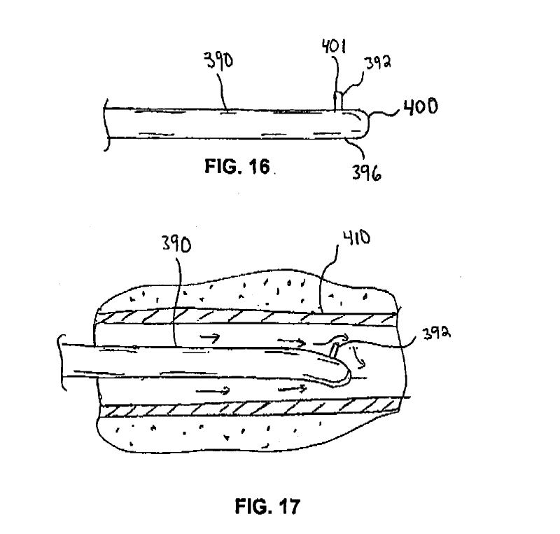

If the elongated catheter 296 is inadvertently guided along an incorrect delivery path, the elongated catheter 296 can be pulled proximally to remove the elongated catheter 296 from the incorrect delivery path. The retracted elongated catheter 296 can then be advanced distally along the correct delivery path. The elongated catheter 296 can be advanced distally and pulled proximally any number of times during a procedure. In some embodiments, the proximal end of the elongated catheter 296 is coupled to an actuation mechanism, for example, an actuation mechanism similar to the actuation mechanism 166 described in connection with FIG. 4. FIGS. 16 and 17 illustrate a wireless elongated catheter 390 including a single steering element 392 coupled at or near a distal tip 396. The wireless elongated catheter 390 is navigated without using a wire, i.e., a guidewire. The illustrated steering element 392 is an outwardly extending tab fixedly coupled to a main body 400 of the elongated catheter 390. The distance between an end 401 of the steering element 392 and the main body 400 can be increased or decreased to increase or decrease the amount of blood that is redirected around the steering element 392, as illustrated in FIG. 17. The steering element 392 can be deployable/retractable to selectively move the main body 400.

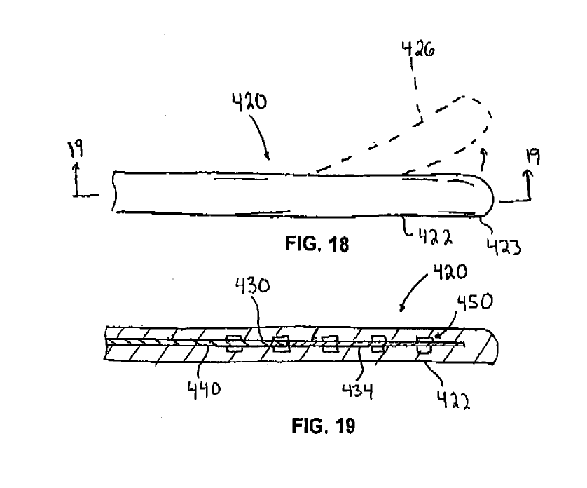

FIG. 18 illustrates a selectively actuatable elongated catheter 420. The elongated catheter 420 includes a distal section 422 selectively movable between a first configuration 423 and a second configuration 426 (illustrated in phantom). The elongated catheter 420, in the first configuration 423, can be substantially straight and suitable for delivery through somewhat linear body vessels. The elongated catheter 420, in the second configuration 426, can be curved in order to navigate through non-linear body vessels. The elongated catheter 420 can move between any number of configurations to permit flexibility when determining an appropriate delivery path to a target site.

Referring to FIG. 19, the distal section 422 includes a light emitter 430 having a selectively actuatable member 434. The actuatable member 434 can move the distal section 422 between the first configuration 423 and second configuration 426. The actuatable member 434 can be made, in whole or in part, of a shape memory material, including, without limitation, a shape memory alloy (e.g., NiTi), a shape memory polymer, a magnetic material, combinations thereof, or other material capable of actively moving between two or more configurations. The shape memory material can be activated by one or more energy sources (e.g., external or internal ultrasound energy sources, heat sources, resistant heaters, and the like). In some embodiments, light sources 450 can generate a sufficient amount of thermal energy to activate the shape memory material. Alternatively or additionally, the actuatable member 434 can be made, in whole or in part, of a piezoelectric material of other electrically deformable materials. The actuator member 434 can thus assume different configurations upon stimulation.

In some embodiments, including the illustrated embodiment of FIG. 19, the actuatable member 434 and a substrate 440 extend in the longitudinal direction along the elongated catheter 420. The light sources 450 are coupled to the actuatable member 434 and the substrate 440.

The elongated catheter 420 can have any number of actuatable members at various positions along its length. For example, the elongated catheter 420 can have a series of longitudinally spaced apart actuatable members. Each actuatable member may have at least two pre-selected configurations. To deliver the elongated catheter 420, a user can select and activate any one of the actuatable members to move the elongated catheter 420 into a desired delivery configuration.

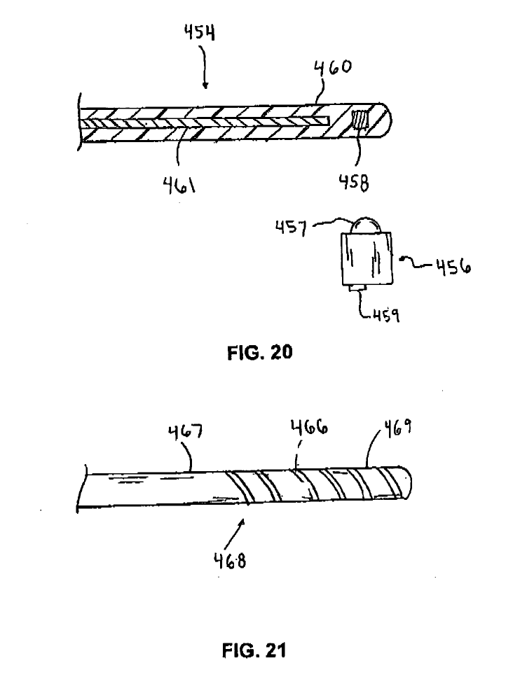

FIG. 20 shows a section of an elongated catheter 454 that is steered using an external steering device 456. A field (e.g., a magnetic field) generated by the steering device 456 (illustrated as a handheld device) can be used to adjust the position of the elongated catheter 454 in situ. The catheter 454 can be pushed, pulled, twisted, bent, or otherwise manipulated using the steering device 456. Because the magnetic field does not traumatize the tissue between the catheter 454 and the steering device 456, the steering device 456 can be used to navigate the catheter 454 through various types of vessels, even vessels that highly susceptible to trauma. As such, the steering device 456 can be conveniently maneuvered externally about the patient to navigate the catheter 454 through different types of tissue.

The illustrated catheter 454 includes a steering element 458 magnetically coupleable to the external steering device 456. One or both of the steering device 456 and steering element 458 can include, without limitation, one or more magnets (e.g., electromagnets, permanent magnets, and the like), ferromagnetic materials, and other devices or materials having suitable magnetic characteristics. In some embodiments, for example, the steering device 456 has an electromagnet 457 and a controller 459 for controlling operation of the electromagnet 457. Current is delivered to the electromagnet 457 to generate a magnetic field that attracts or repels the steering element 458. In this manner, the catheter 454 can be laterally deflected a desired amount. The characteristics of the magnetic field, properties of the steering element 458, and the like can be selected based on the distance between the desired delivery path for the catheter 454, desired steerability, and the like.

The steering element 458 can be a magnetic bead or other type of magnetic element for generating a magnetic field. As shown in FIG. 20, the steering element 458 is embedded in a main body 460 of the elongated catheter 454 and, in some embodiments, may be incorporated into a light source 461. In other embodiments, the steering element 458 is coupled to an external surface of the main body 460. For example, the steering element 458 can be a magnetic or ferromagnetic cylindrical member mounted on the exterior of the main body 460. The position and number of steering elements 458 can be selected based on the desired steering capabilities. For example, a plurality of selectively activatable steering elements (e.g., electromagnets) can be longitudinally spaced along the catheter 454, or provided in any other desired orientation or positions. The activatable steering elements can be concurrently or sequentially activated to magnetically couple different regions of the catheter 454 to the steering device 456.

Referring to FIG. 21, a steering element 466 in the form of a helical electromagnet surrounds and extends along a section of a main body 467 of an elongated catheter 468. The steering element 466 can be coupled (e.g., bonded, adhered, partially embedded, and the like) to an exterior surface 469 of the main body 467. In other embodiments, the steering element 466 is incorporated into the main body 467. To protect the steering element 466, the steering element 466 can be integrated into an internal light source, such as a light bar. A single power supply can power both the light source and the steering element. The steering element 466 can be a coated conductive trace, magnetized wire, or other suitable device for interacting with a magnetic field.

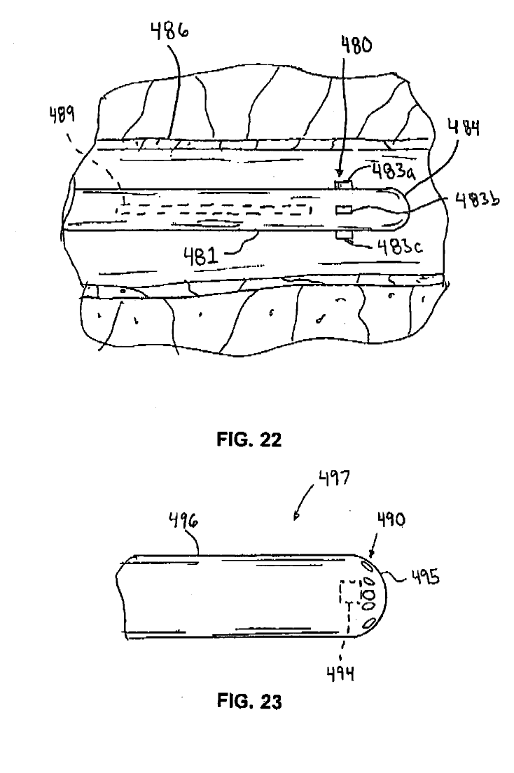

FIG. 22 shows a propulsion system 480 that expels and/or draws in the bodily fluid to navigate an elongated catheter 481. Pumping action, pulsing action, or other types of action can be provided by the propulsion system 480. For example, the propulsion system 480 can pulse bodily fluid to drive the elongated catheter 481 along a section of a vessel 486 to be treated, even if there is substantially no natural flow of bodily fluid. Thus, the elongated catheter 481 may be steered through remote peripheral vessels that provide minimal amounts of natural fluid flow.

The illustrated propulsion system 480 includes a plurality of pumping devices 483a, 483b, 483c (collectively 483) for actively pumping or pulsing the bodily fluid to distally advance a tip 484 through the vessel 486. Each pumping device 483 can include, without limitation, a microfluidic chip, micro-electro-mechanical systems (MEMS) pump, or the like. Power is delivered to the pumping devices 483 via circuitry extending along the length of the elongated catheter 481. The pumping devices 483 can pump fluid in the proximal direction, distal direction, or both. To reduce or prevent unwanted trauma to tissue, the steering force can be generated by low pressure, high volume flows from the pumping devices 483. The pumping devices 483 can also output high pressure, low volume flows. Other types of fluid flows are also possible based on the designed fluidic components, desired interaction with the tissue, or the like.

A light source 489 (shown in phantom) and the pumping devices 483 can be activated using the same signal or different signals. For example, the same signal can drive both the light source 489 and the pumping devices 483 to ensure concurrent light delivery and pumping. The number of pumping devices and their locations can be selected based on the desired steerability, properties of the body fluid (e.g., viscosity), and the like.

Propulsions systems can also be incorporated into a main body of an elongated catheter. FIG. 23, for example, shows a main body 496 having a plurality of ports 490 through which bodily fluid (e.g., blood) flows. A propulsion system 494 (shown in phantom line) is proximate the tip 495 and causes fluid flow inwardly and/or outwardly through the ports 490, which can be at various locations along the main body 496. For example, the ports 490 can be radially adjacent an internal light source in the main body 490. Saline or other biocompatible fluids can also be outputted from the ports 490 to deflect the main body 496.

Additionally or alternatively, a drug can be delivered through the ports 490. Different ports may be used for drug delivery and propulsion. Drugs can thus be administered directly to the target tissue to avoid unwanted drug delivery to remote tissue. Of course, other light delivery systems disclosed herein can also have one or more integral drug delivery ports.

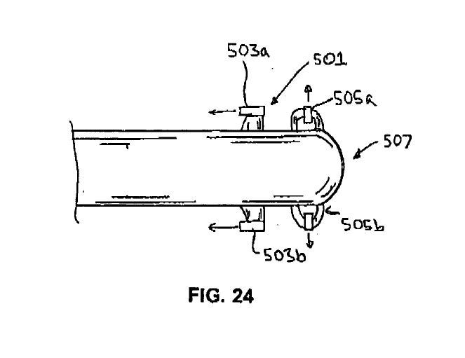

FIG. 24 shows a propulsion system 501 that includes one or more pumping devices 503a, 503b for axial motion and one or more pumping devices 505a, 505b for off-axis motion. The pumping devices 503a, 503b, 505a, 505b can be similar or identical to the pumping devices 483 discussed in connection with FIG. 22. These devices are mounted on the exterior of a catheter 507. Accordingly, various types of propulsion systems can be used alone, or in combination, to achieve the desired steerability, including moving distally, moving proximally, displacing laterally, rotating, twisting, and the like.

The light delivery systems disclosed herein can have any number of catheters. A light delivery system, in some embodiments, may include more than two catheters. Each of the catheters can be positioned within the subject so as to generate a substantially uniform light field. The catheters can be positioned within and/or near the target tissue. For example, if a solid tumor is to be treated, at least one catheter can be delivered into the solid tumor. At least one catheter can be positioned external to the solid tumor. In this manner, multiple catheters can thoroughly illuminate the tumor to ensure that the tumor is properly treated.

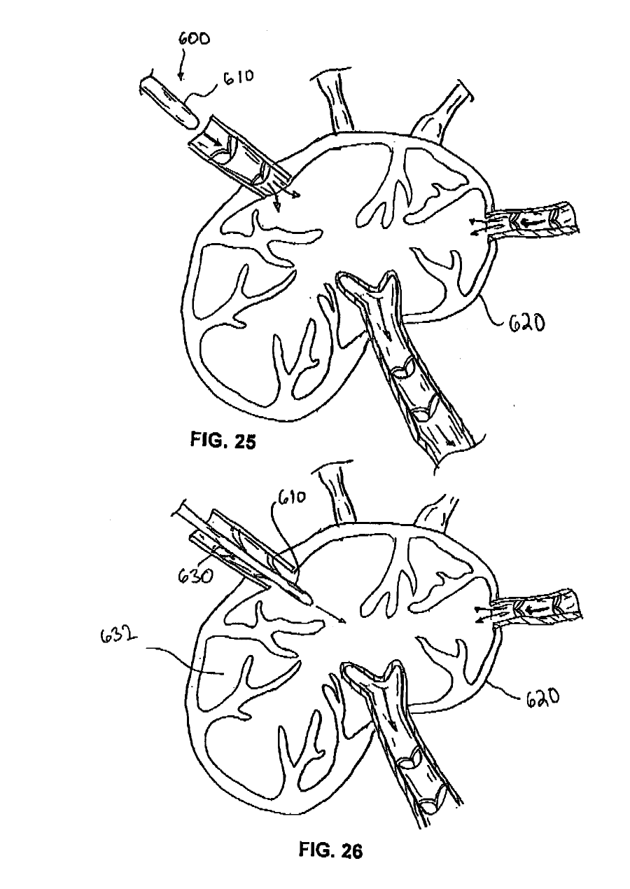

FIG. 25 shows an elongated catheter 600 for performing light therapy on the lymphatic system. The elongated catheter 600 has a distal section 610 configured and dimensioned to pass through a body vessel that provides access to a lymph node 620. The catheter 600 can be deployed intravascularly to affect lymph nodes that are adjacent or proximate to vascular vessels.

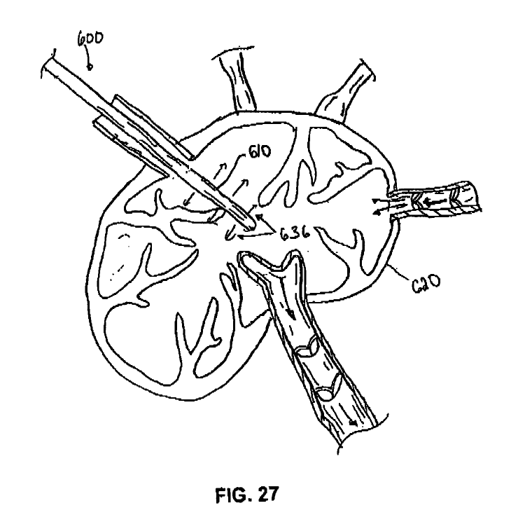

Because the elongated catheter 600 has a relatively low-profile, the distal section 610 can be passed through an afferent or efferent lymphatic vessels. FIG. 26 shows the distal section 610 passing through a lymphatic vessel 630 and into a cortex 632 of the node 620. Once a desired portion of the distal section 610 is disposed within the cortex 632, a light emitter of the distal section 610 can be energized. The energized light emitter delivers light, illustrated by the arrows 636 of FIG. 27, to targeted lymphatic tissue. If the node 620 consists of cancerous tissue, or other types of unwanted tissue, the distal section 610 can be conveniently navigated within range of that unwanted tissue. If the distal section 610 includes a port for drug delivery, a drug can be delivered to unwanted tissue after the port is positioned within the node 620.

Cancer, infections, allergies, autoimmunity problems, and other unwanted conditions can be associated with the lymphatic system. One or more lymph nodes, or other lymphatic tissue, may require stimulation, reduction, destruction, and/or removal, as well as other types of therapy to treat the condition. Light therapy for node reduction is especially useful to treat cancerous nodes, enflamed or enlarged nodes (which may be painful), and/or lymphatic tissue causing unwanted conditions (e.g., blockage such as blockage of the tonsils or adenoids), as well as to improve pathologically immunity, for example, to treat multiple sclerosis. Lymph node stimulation can be used to treat different types of infection, cancer, or the like.

Stimulated lymph nodes may increase the destruction rate of cancer cells. Destruction of cancer cells can in turn lead to activation of a tumor-specific immune response. Without being bound by theory, light therapy is thought to cause destruction of tumors leading to an antigen cascade whereby tumor antigens from the destroyed tumor cells are presented to T cells by a variety of cells including macrophages and dendritic cells (See e.g., C. Kudo-Saito, et al., Clin Cancer Res. 2005 Mar. 15; 11(6):2416-26; Pilon S A, et al. 2003 J Immunol; 170:1202-8; Markiewicz M A, et al. Int Immunol 2001; 13:625-32; Cavacini L A, et al. Clin Cancer Res 2002; 8:368-73; Butterfield L H, et al. Clin Cancer Res 2003; 9:998-1008). In this manner, tumor-specific epitopes not previously available to the immune system, are exposed and activate the immune system to recognize the tumor cells. This tumor antigen priming of the immune system may occur directly at the site of tumor destruction, in the draining lymph nodes or may occur in the blood. In certain cases, it may be possible to detect a specific tumor antigen signature after light therapy of the invention as described herein. Such tumor antigen signatures may in turn be used in diagnosis and/or for monitoring therapy of cancers. Thus, the light therapy of the invention provides the added benefit of activating the immune system against the tumor cells.

In certain embodiments, the light therapy of the invention can be used in conjunction with tumor vaccine strategies to further activate the immune system. Any of a variety of tumor vaccine strategies known to the skilled artisan can be combined with the therapy of the present invention (see e.g., Disis M L, et al. Lancet. 2009 Feb. 21; 373(9664):673-83; Rogers L J, et al. Curr Opin Oncol. 2008 September; 20(5):570-4; Fournier P and Schirrmacher V Expert Rev Vaccines. 2009 January; 8(1):51-66; see also clinicaltrials.gov). Because the light delivery system 600 preserves the lymph channels, the lymphatic system can continue to function, thereby limiting, or substantially preventing lymphedema, sequelae, pain, skin breakdown, limb swelling, and/or secondary infections.

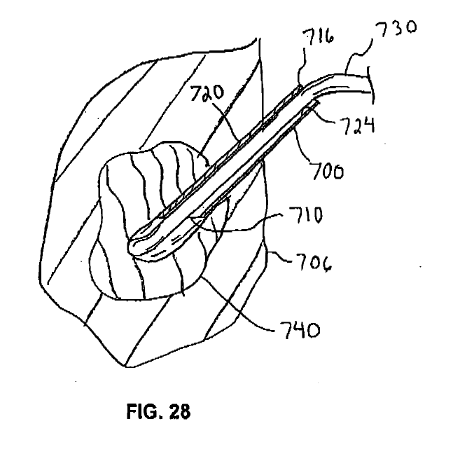

FIG. 28 shows one method of delivering and deploying a light delivery system 730. An insertion device 700 is inserted into tissue 706 of the patient. The insertion device 700 has an insertion end 710, a loading end 716, and a main body 720 extending therebetween. A working lumen 724 extends between the insertion end 710 and the loading end 716. Once the insertion device 700 is placed in the subject, the light delivery system 730 is moved through the loading end 716 and into the working lumen 724. The light delivery system 730 is then advanced through the working lumen 724 towards a target site 740. Because the light delivery system 730 can have a low-profile, a correspondingly low-profile insertion device 700 can be employed to limit trauma to the subject. For example, the insertion device 700 can be an insertion needle for providing access to a lymph node.

After performing light therapy, the light delivery system 730 of FIG. 28 can be pulled proximally through the working lumen 724. The insertion device 700 can be withdrawn from the tissue 706 and removed from the subject.

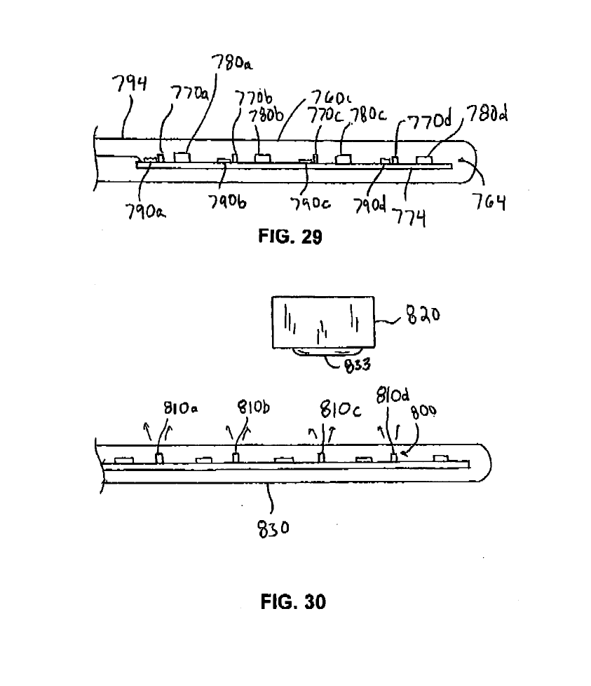

FIG. 29 shows a portion of a light delivery system 760 including a visualization system 764 that can be used before, during, and/or after performing light therapy. During catheter delivery, the visualization system 764 is used to steer through hollow body vessels or around organs to reduce, limit, or substantially prevent unwanted injury or trauma to the subject's tissue. Once the light delivery device 760 is near the target site, the visualization system 764 is used to precisely locate a light emitter with respect to the target tissue.

Because the visualization system 764 is incorporated into the light delivery system 760, the visualization system 764 can assess the patient while in situ. The visualization system 764 can help evaluate (e.g., provide 360° viewing of the targeted tissue) the light therapy in real-time to provide user feedback related to the progress of the effect of the light therapy. Various measurable parameters, such as characteristics of tissue (e.g., optical characteristics), water content, blood flow, echogenicity, fluorescence, contrast enhancement, and the like may be ascertained using the visualization system 764. Real-time assessment allows for a tailored and modulated treatment program.

If treatment agents are used, the ongoing photo activation effect can be monitored and adjusted by controlling various operating parameters, such as light wavelength, light intensity, position of the light delivery system, amount of treatment agent used, period and frequency of treatment cycles, and the like. The visualization system 764 can also be used to monitor healthy untargeted tissue. In some embodiments, for example, the normal tissue at a tumor interface may be monitored to detect the adequacy and accuracy of the light therapy at the interface.

With continued reference to FIG. 29, the visualization system 764 includes a plurality of optical sensors 770a-d and a plurality of light sources 780a-d coupled to a substrate 774. The light sources 780a-d illuminate tissue activated with a treatment agent and cause an optical response, such as fluorescence of the treatment agent. The optical sensors 770a-d can detect the fluorescence, which may indicate treatment effect, zone of effect, or photo bleaching, as well as other characteristics related to the light therapy. The treatment program can be modified or adjusted based on signals from the sensors 770a-d.

In some embodiments, including the illustrated embodiment of FIG. 29, the optical sensors 770a-d can be photodiodes that are configured to measure one or more selected wave lengths or wavebands. The photodiodes 770a-d have optical filters that block one or more wavelengths or wavebands. For example, an optical filter of the photodiode 770a can let a target wavelength, such as 760 nm, pass therethrough while blocking other wavelengths. The target wavelength can correspond to the fluorescence wavelength of the treatment agent.

In the illustrated embodiment of FIG. 29, a plurality of light sources 790a-d are also mounted to the substrate 774. Each of the light sources 790a-d is located in proximity to a respective optical sensor 770a-d. Tissue illuminated by the light sources 790a-d can be evaluated by the optical sensors 770a-d. For example, the light sources 790a-d can emit blue light detectable by the optical sensors 770a-d. The light sources 780a-d can emit red light suitable for performing the light therapy. The red light sources 780a-d can be turned off and the blue light sources 790a-d can be activated to measure a fluorescent signal from the administered treatment agent. The stokes shift from blue excitation to red fluorescence may be sufficient to predict or measure an accurate signal to noise ratio, or other parameters of interest. The blue light sources 790a-d can be used to perform therapy as well, if less tissue penetration is preferred.

The visualization system 764, in some embodiments, can employ spectroscopic techniques. The light sources 790a-d can be infrared emitters and the optical sensors 770a-d may detect infrared light. For example, the optical sensors 770a-d can measure the spectral band from the fluorescence of the treatment agent. Signals from the optical sensors 770a-d are transmitted to a control system.

The optical sensors 770a-d can include at least one waveguide (e.g., a fiber optic) that is embedded in a main body 794. The optical sensors 770a-d can transmit emission signals via the waveguide to a control system that has a spectra meter, such as a miniature grating spectra meter capable of separating the spectrum and providing real time feedback.

FIG. 30 illustrates a system 800 for communicating with an external system 820. The system 800 includes a plurality of emitters 810a-d capable of outputting signals or energy that can be sensed using another system. The emitters 810a-d can be light emitters, such as IR emitters. In such embodiments, the energy emitters 810a-d emit a sufficient amount of light such that an optical sensor 833 of the external system 820 receives a measurable amount of the emitted light. The external system 820 can be capable of measuring energy emitted from the energy emitters 810a-d, especially when the energy emitters 810a-d are adjacent the target site. The target site can be interposed between the implanted distal section 830 of the elongated catheter and the optical sensor 820.

A controller can correlate a change in signals from the optical sensor 833 with a treatment effect associated with light therapy being performed. The controller can modulate the emitted signal intensity, frequency of treatment, treatment duration, or combinations thereof to gain additional information about the patient, such as blood flow, transmissivity of tissue, and the like.

The external system 820 of FIG. 30 can be a handheld system suitable for placement against a subject's skin. The optical sensor 833 may comprise, without limitation, one or more IR receivers, mercury-cadmium-telaride (MCT) detectors, paralytic detectors, thermopiles, bolometers, or silicon micro bolometers, as well as other suitable components for detecting or sensing electromagnetic energy.

In some embodiments, the emitters 810a-d can be encodable objects with information and capable of outputting one or more signals that are receivable by the external system 820. For example, the emitters 810a-d can be radio frequency identification tags that may take the form of radio frequency identification (RFID) circuits, transponders, devices, or tags. The emitters 810a-d can communicate with the external system 820 in the form of a reader. The term “reader” is broadly construed to include, without limitation, verifiers, interrogators, controllers, read elements, or other devices used to receive information from the emitters 810a-d.

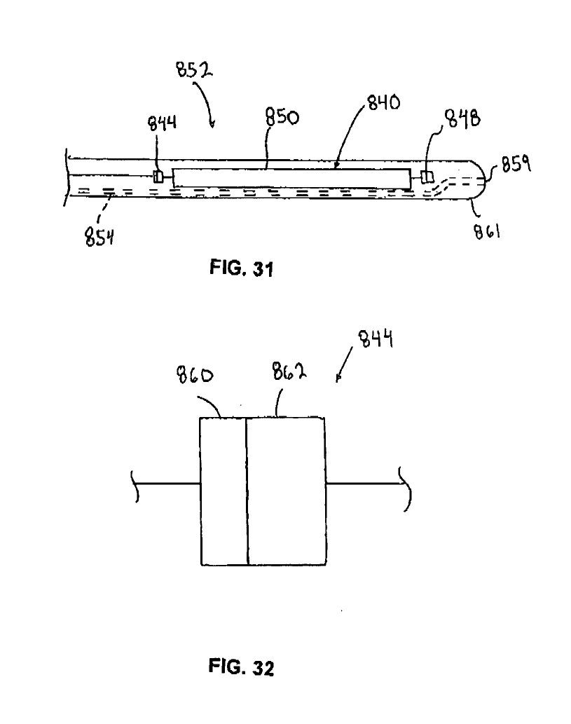

FIG. 31 illustrates a visualization system 840 including a first emitter/sensor 844 and a second emitter/sensor 848. When a light emitter 850 illuminates tissue, the first emitter/sensor 844 and second emitter/sensor 848 evaluate tissue and, in some embodiments, can map changes in the targeted or untargeted tissue to predict or determine, among other things, a zone of treatment. The visualization system 840 can be used to predict an overall zone of treatment, based on the light delivery system illuminating a certain volume of tissue (e.g., a known or estimated volume of tissue). The shape of illuminated tissue can be related to the optical characteristics of the tissue. The elongated catheter 852 can be advanced distally or proximally to reposition the light bar 850 with respect to the target tissue based on the progress of the light therapy using the predicted zone of treatment.

The elongated catheter 852 also includes at least one longitudinally extending working lumen 854 (shown in broken line) for receiving a guidewire. In some embodiments, a fluid, such as an infusion fluid, can be delivered through the working lumen 854 and a port 859. The port 859 can be at a distal tip 861 of the elongated catheter 852.

FIG. 32 shows the first emitter/sensor 844, including an optical sensor 860 and an emitter 862. The emitter 862 can be activated continuously, intermittently, or by using any program to evaluate the tissue, if needed or desired. The various embodiments described above can be combined to provide further embodiments.

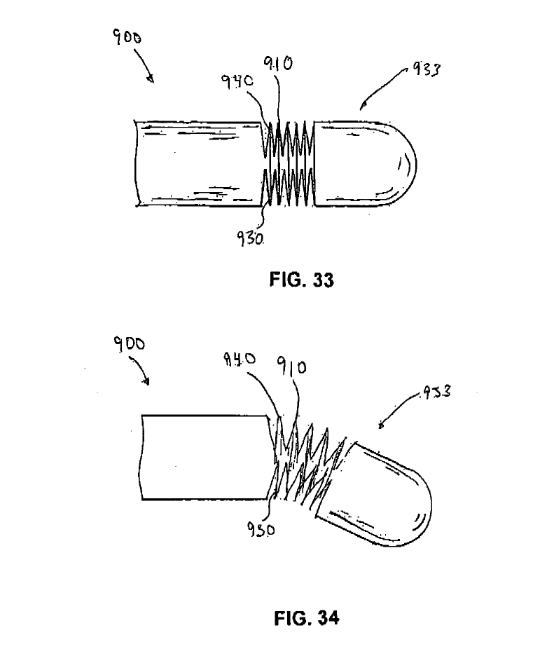

FIG. 33 shows a light delivery catheter 900 with a flexible structure 910. The flexible structure 910 can be an accordion portion that provides localized deformation and includes pleated bellows. Different sections of the bellows can be isolated from one another for independent inflation or deflation. The flexible structure 910 can assume different configurations to move a distal section 933 to different curved configurations.

The illustrated flexible structure 910 includes five pleated sections that can be in fluid communication with an external pump. The external pump can inflate/deflate the pleated sections. For example, a proximal most pleated section 930 can include a plurality of chambers in fluid communication with corresponding inflation lumens extending proximally to the external pump. Selected chambers can be pressured to expand an upper portion 940 of the section 930. Each of the upper portions of the pleated sections can be inflated in this manner until the delivery catheter 900 is in a curved configuration, as shown in FIG. 34. In some embodiments, one or more longitudinally-extending lumens can be pressurized to straighten that section of the flexible structure 910, thus curving the distal section 933 away from the pressurized lumens.

The flexible structure 910 can be made, in whole or in part, of silicon, rubber, polymers, or other biocompatible and flexible materials. The structure 910 can also have other segmented, pleated, or hinged configurations.

In various embodiments, one or more inflation/deflation lumens, pull wires, stylets, stiffeners, biasing members, inflatable members, or the like can be used to move the distal section 933 in order to achieve a wide range of complex configurations.

All of the U.S. patents, U.S. patent application publications, and U.S. patent applications referred to in this specification and/or listed in the Application Data Sheet, are incorporated herein by reference, in their entirety. Aspects of the embodiments can be modified, if necessary to employ concepts of the various patents, applications, and publications to provide yet further embodiments. The catheters can be modified to control the direction of emitted light or to achieve a desired light distribution. Catheters that output latterly directed light are well suited for treating tissue surrounding vessel walls. Catheters that output light in a distal direction are well suited for treating tissue at other locations. U.S. application Ser. No. 10/799,357 (corresponding to U.S. Publication No. 20050228260) discloses various types of light sources, light source mounting arrangements, distal tip configurations, and the like that can be incorporated into the embodiments disclosed herein in order to direct light energy in a desired direction. U.S. application Ser. No. 10/799,357 is incorporated by reference herein in its entirety.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An intraluminal system for performing light therapy, comprising:
an elongated catheter including a proximal section, a distal section, and a central section between the proximal section and the distal section, the central section and the distal section being configured and dimensioned to be delivered through a lumen of a peripheral vessel, and the distal section including at least one light emitter operable to receive electrical energy to generate a sufficient amount of light to perform light therapy on tissue adjacent to the peripheral vessel.

2. The intraluminal system of claim 1, wherein the elongated catheter further includes an imaging element.

3-7. (canceled)

8. The intraluminal system according to claim 1, further comprising:
a lubricious coating on an outer surface of the elongated catheter.

9. The intraluminal system according to claim 1, further comprising:
an optical sensor physically coupled to the elongated catheter.

10. The intraluminal system of claim 9, further comprising:
a controller connected to the optical sensor, and the controller being configured to modulate energy emitted from the at least one light emitter based on at least one signal from the optical sensor.

11-15. (canceled)

16. The intraluminal system according to claim 1, wherein the distal section has a cross-sectional area and the at least one light emitter is capable of outputting energy having an energy density, and wherein a ratio of the energy density to the cross-sectional area of the distal section is greater than about 1,000 mW/(cm2)2.

17. The intraluminal system according to claim 1, wherein the distal section is configured and dimensioned to pass through a lumen of a peripheral vascular vessel within a solid tumor.

18-26. (canceled)

27. The intraluminal system according to claim 1, wherein the distal section has an average outer diameter that is equal to or less than about 0.1 mm.

28. The intraluminal system according to claim 1, wherein the distal section is dimensioned to pass through a lumen of a lymphaticvessel connected to a lymph node.

29. The intraluminal system according to claim 1, wherein the distal section is dimensioned to fit in a lymph node, and the at least one light emitter is capable of outputting a therapeutically effective amount of light for treating tissue of the lymph node while the distal section is positioned within the lymph node.

30. (canceled)

31. (canceled)

32. The intraluminal system according to claim 1, wherein the distal section includes a flexible structure configured to assume different configurations to move the distal section to different curved configurations.

33. A light delivery system, comprising:
a first catheter including a first light emitter and a first sensor;
a second catheter including a second light emitter and a second sensor, the first sensor is capable of detecting light emitted by the second light emitter, and the second sensor is capable of detecting light emitted by the first light emitter; and
a control system configured to control the first light emitter based on a signal from the second sensor and to control the second light emitter based on a signal from the first sensor.

34. The light delivery system of claim 33, wherein the first catheter and the second catheter are physically coupled to the control system and independently deliverable.

35. The light delivery system according to claim 33, wherein the first light emitter has at least one light source.

36. The light delivery system according to claim 33, wherein the first sensor is a photodiode or an IR detector.

37. The light delivery system according to claim 33, wherein the control system is configured to sequentially activate the first and second light emitters so as to activate a substantial portion of a photoactive agent between the first and second catheters.

38. The light delivery system according to claim 33, wherein the first catheter has a distal section that includes the first light emitter, and the distal section has an average outer diameter that is equal to or less than about 1 mm.

39. An intraluminal catheter for performing light therapy on a lymph node, comprising:
a central section configured for placement in a subject; and
a distal section coupled to the central section, the distal section including at least one light source capable of outputting light for performing light therapy, the distal section being configured and dimensioned for delivery through a lumen of a lymphatic vessel to position the at least one light source adjacent lymphatic tissue within the lymph node.

40. The intraluminal catheter of claim 39, wherein the distal section has an average diameter less than about 200 μm.

41. The intraluminal catheter according to claim 39, wherein the distal section is dimensioned to be delivered percutaneously through a lymphatic system into the lymph node.

42. The intraluminal catheter according to claim 39, wherein the distal section is dimensioned to be delivered through a vessel having a lumen with a diameter that is less than about 200 μm.

43. The intraluminal catheter according to claim 39, wherein the at least one light source emits a sufficient amount of light to activate a therapeutically effective amount of a photosensitive agent in the lymph node.

44. The intraluminal catheter according to claim 39, wherein the central section has a length sufficient to permit percutaneous delivery of a distal tip of the distal section into the lymph node.

45. The intraluminal catheter according to claim 39, further comprising:
a port at the distal section and an infusion lumen extending proximally from the port through the central section.

46-65. (canceled)

66. A method of treating lymphatic tissue of a subject, the method comprising:
moving a catheter along a body lumen towards lymphatic tissue;
advancing a distal tip of the catheter through the body lumen into the lymphatic tissue; and
activating a light emitter of the catheter to deliver light to the lymphatic tissue adjacent the light emitter.

67. The method of claim 66, further comprising:
placing a delivery needle in the subject, the delivery needle having a working lumen; and
advancing the catheter through the working lumen of the delivery needle into the body lumen.

68. The method according to claim 66, wherein advancing the distal tip of the catheter includes selectively actuating the distal tip between a first configuration and a second configuration.

69. The method according to claim 66, wherein the lymphatic tissue surrounds the light emitter when the light emitter is activated.

70. The method according to claim 66, further comprising:
administering a treatment agent to the subject such that a therapeutically effective amount of the treatment agent in the lymphatic tissue is activated by the light emitter.

71. The method according to claim 66, wherein the light emitter is activated a sufficient length of time to effectively stimulate functioning of the lymphatic tissue.

72-77. (canceled)
